(19) United States
(12) Patent Application Publication
(10) Pub. No.: US 2009/0252716 A1
(43) Pub. Date: Oct. 8, 2009

(54) METHODS FOR IDENTIFYING NUCLEAR RECEPTOR/LIGAND COMBINATIONS FOR TARGETING BRAIN TUMOR STEM CELLS AND FOR THEIR USE

(75) Inventor: John J. Bright, Carmel, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: Clarian Health Partners, Inc. d/b/a Methodist Research Institute, Indianapolis (IN)

(21) Appl. No.: 12/417,689

(22) Filed: Apr. 3, 2009

Related U.S. Application Data
(60) Provisional application No. 61/042,356, filed on Apr. 4, 2008.

(51) Int. Cl. A61K 31/7076; C12Q 1/02; A61K 31/4045; A61K 31/496; A61K 31/429; A61K 31/195; A61K 31/5575; A61P 35/00

(52) U.S. Cl. 424/94.1; 435/29; 514/415; 514/254.11; 514/368; 514/567; 514/573

(57) ABSTRACT

An in vitro method is provided for identifying nuclear receptors abnormally expressed by brain tumor stem cells and a corresponding ligand which, if administered to brain tumor stem cells (BTSC's), is capable of inhibiting cell proliferation. Once the nuclear receptor/ligand combination has been identified, it can be utilized in vitro and in vivo to inhibit the proliferation and survival of the cancerous stem cells and ultimately affect proliferation and survival of tumors. The method can be utilized alone or in combination with other treatment methods. The method can also be utilized with regard to other forms of cancer which have cancerous stem cells associated therewith and which abnormally express one or more nuclear receptors.

### REFERENCE TO RELATED APPLICATIONS

The present application claims the benefit of U.S. Provisional Patent Application No. 61/042,356, filed Apr. 4, 2008, entitled METHOD FOR IDENTIFYING NUCLEAR RECEPTOR/LIGAND COMBINATIONS USEFUL FOR TARGETING BRAIN TUMOR STEM CELLS AND FOR THE USE OF THE COMBINATION IN TREATING BRAIN TUMORS, which is hereby incorporated herein by reference in its entirety.

### BACKGROUND

Brain tumors are devastating cancers that present unique challenges to therapy and pose major health problems throughout the world. Glioblastoma is the most frequent primary malignant brain tumor in adults. For individuals inflicted with Glioblastoma, median survival is generally less than one year from the time of diagnosis, and even in the most favorable situations, most patients die within two years.

Standard therapy consists of surgical resection to the extent that is safely feasible, followed by radiotherapy and chemotherapy, which have significant side-effects and limited efficacy. Recent advances have led to the development of targeted molecular therapies with some improvement in therapeutic efficacy and reduced toxicity. Despite recent advances in surgery, radiation, chemotherapy and the targeted molecular therapies, a cure for brain tumors remains elusive. The multi-drug resistance and fast recurrence of tumors are some of the current challenges in combating brain tumors.

Neural stem cells (NSC) are a small population of resident cells in the CNS, capable of migration, growth and differentiation into neuron-glial cells. Properly functioning NSC can mediate CNS renewal and repair damage caused by injury, disease and the like. Shortly after their identification, it was recognized that NSC's could be important in treating a variety of neurodegenerative diseases. Studies have shown that the progenitor cells of the adult mouse sub ventricular zone proliferate, migrate and differentiate into oligodendrocytes after demyelination. Under optimum culture conditions, the NSC grows and differentiates into neuro-glial cells in vitro. Adoptively transferred NSC's migrate, grow and differentiate into neuro-glial cells in normal brain, suggesting their use in the treatment of neurodegenerative diseases. Recent studies have shown promise in adoptive transfer of cultured neural stem cells for the treatment of many neurodegenerative diseases including multiple sclerosis.

NSC's may yet become an important tool for restoring defective cells and functions of the CNS and for treating a variety of diseases of the brain. However, cancerous NSC can be a source of brain tumors. Recent studies have identified tumor stem cells in brain that are responsible for highly invasive and aggressive tumors which are resistant to chemo/radio-treatment. Such tumors tend to reoccur following conventional treatment. While NSC's continue to show promise in treating a variety of brain diseases, it is believed that resident cancerous stem cells also are a potential source of brain tumors. Evidence for the existence of cancer stem cells has been suggested for hematological malignancies and, more recently, for certain solid tumors, such as breast, prostate, colon cancer and brain tumors. The failure to cure many forms of cancer may be due to the fact that typical therapies target rapidly proliferating tumor cells, which respond transiently, while sparing the tumor stem cells, which tend to be momentarily latent but have high tumorigenic potential.

The identification of compositions which can target brain tumor stem cells (BTSC) and are safe to the individual being treated is important to the development of comprehensive treatment for brain tumors. Such a comprehensive method would need to treat not only the rapidly forming tumor cells, but additionally target BTSC's. Thus, new therapeutic agents are needed for the treatment of brain tumors and related forms of cancer that target not only the rapidly growing cancer cells, but which can also target the more latent BTSC's. Only by incorporating such an approach can drug resistance and tumor recurrence be avoided and improved survival rates obtained.

### SUMMARY

Nuclear receptors are a family of ligand-dependent transcription factors that mediate responses to steroids, retinoids, thyroid hormones, vitamin D, and PPAR ligands. Nuclear receptors play key roles in embryonic development and regulation of immune and inflammatory responses. Nuclear receptors contain a ligand binding domain and a DNA binding domain. Upon activation with specific ligands, the nuclear receptors heterodimerize and bind to a series of cofactors leading to the activation or repression of target genes.

As will become apparent from the following discussion, the present disclosure provides methods for: (a) identifying nuclear receptors having an altered expression by cancerous stem cells, (b) determining nuclear receptor/ligand combinations having the ability to inhibit the survival of BTSC's and (c) utilizing the combinations to inhibit proliferation and survival of BTSC's in vitro and in vivo. The methods disclosed herein are applicable to other forms of cancer having stem cells similarly having an altered expression of specific nuclear receptors, and can be effectively utilized to treat mammalian forms of cancer in vivo.

A first aspect of the present disclosure involves a method for the treatment of a tumor. This method involves obtaining a sample of tumor cells from a subject possessing the tumor; isolating tumor stem cells from the sample of tumor cells; analyzing the tumor stems cells for expression levels of nuclear receptors; identifying a nuclear receptor which has the expression level; identifying an agonist for the nuclear receptor; and treating the subject with said agonist. This method is particularly effective in the treatment of a brain tumor. Certain preferred embodiments of this method involve identifying a nuclear receptors selected from the group consisting of HNF4α, PNR, TLX, RORα, RORγ, PXR, LXRβ, LXR, PPARγ, DAX1, and CARα. Other preferred embodiments of this method involve identifying a nuclear receptors selected from the group consisting of HNF4α, PNR, TLX, PXR, LXRβ, LXR, DAX1, and CARα. Still other preferred embodiments of this method involve identifying a nuclear receptor selected from the group consisting of HNF4α, RORα, RORγ, PXR, LXRβ, LXR, PPARγ, and CARα. Finally, certain preferred embodiments of this method involve identifying a nuclear receptor selected from the group consisting of HNF4α, PXR, LXRβ, LXR, and CARα. Preferred embodiments of this method involve treating the subject with an agonist selected from the group consisting of Butyryl CoA, CITCO, GW3965, Melatonin, Rifampicin, and 15d-PGJ2. Treatment according to this method involves placing the identified agonist at the site of the tumor.

A further aspect of the present disclosure involves a method for the treatment of a subject with a brain tumor which contains brain tumor stem cells. The method involves identifying a nuclear receptor expressed by the brain tumor stem cells; identifying an agonist of the nuclear receptor expressed by brain tumor stem cells; and treating the subject with the agonist. A preferred embodiment of this method involves identifying a nuclear receptor selected from the group consisting of HNF4α, PNR, TLX, RORα, RORγ, PXR, LXRβ, LXR, PPARγ, DAX1, and CARα. A further preferred embodiment of this method involves identifying a nuclear receptor selected from the group consisting of HNF4α, RORα, RORγ, PXR, LXRβ, LXR, PPARγ, and CARα. When the nuclear receptor is identified from the preceding group, preferred agonists are typically selected from the group consisting of Butyryl CoA, CITCO, GW3965, Melatonin, Rifampicin, and 15d-PGJ2. Another preferred embodiment of this method involves identifying a nuclear receptor selected from the group consisting of consisting of HNF4α, PXR, LXRβ, LXR, and CARα. When the nuclear receptor is identified from the preceding group, preferred agonists are typically selected from the group consisting of Butyryl CoA, CITCO, GW3965, and Rifampicin. For the various embodiments described, an effective method of treating the tumor is to place the agonist of the nuclear receptor at the site of the tumor.

A further aspect of the present disclosure involves an in vitro method of identifying a substance to treat tumors. The method involves obtaining a sample of tumor stem cells from a subject possessing a tumor; identifying in the sample of tumor stem cells an expression level of a nuclear receptor; and identifying a ligand of the nuclear receptor identified in the sample of tumor stem cells wherein the ligand is the substance identified to treat the tumor. This method is particularly useful for identifying substances to treat a brain tumor.

A further aspect of the present disclosure involves an in vitro method for identifying nuclear receptor/ligand combinations which can be utilized to target cancerous stem cells. The method involves identifying a nuclear receptor expressed by the cancerous stem cell; selecting a ligand expected to coordinate with the selected nuclear receptor; culturing the cancerous stem cells in the presence of the selected ligand; and observing the level of inhibition obtained in the presence of the selected ligand, wherein an inhibition of the cancerous stem cells indicates that the ligand can be utilized to target the cancerous stem cells. This method is particularly useful for identifying substances to target cancerous brain tumor stem cells.

A further aspect of the present disclosure involves an in vivo method of treating brain tumor stem cells. This method involves identifying a patient with a brain tumor possessing brain tumor stem cells; and administering to the identified patient a material from the group consisting of Butyryl CoA, CITCO, GW3965, Melatonin, Rifampicin, and 15d-PGJ2.

A further aspect of the present disclosure involves administering to the identified patient a material from the group consisting of Butyryl CoA, CITCO, GW3965, and Rifampicin. The material can be administered by methods selected from the group consisting of injection, osmotic pump, IV administration, ingestion, dermal application, inhalation and the like.

A further aspect of the present disclosure involves an in vivo method for treating a mammalian organism having a cancerous growth or tumor. This method involves identifying a ligand associated with a nuclear receptor having altered expression in a cancerous stem cell obtained from the organism and administering a pharmaceutical formulation containing the ligand to the organism. The pharmaceutical formulation can be administered by methods selected from the group consisting of injection, osmotic pump, IV administration, ingestion, dermal application, inhalation and the like. Preferred embodiments of this method involve pharmaceutical formulations containing a ligand selected from the group consisting of Butyryl CoA, CITCO, GW3965, Melatonin, Rifampicin, and 15d-PGJ2. More preferred embodiments of this method involve pharmaceutical formulations containing a ligand selected from the group consisting of Butyryl CoA, CITCO, GW3965, and Rifampicin.

A still further aspect of the present disclosure involves a method for identifying nuclear receptors having an altered expression in a form of cancerous stem cells. This method involves obtaining a culture of regular cancer cells; obtaining a culture of the related cancerous stem cells from the regular cancer cells; culturing both sets of cells separately, determining a level of expression of a selected nuclear receptor in both cultures; and comparing the level of expression of the selected nuclear receptor by the cancerous stem cell compared to the regular cancer cells wherein a difference in the level of expression indicates the nuclear receptor having an altered expression. This method is particularly applicable to Glioblastoma cells.

### FIGURES

FIG. 1 illustrates U87MG and T98G BTSC tumorspheres and the glioblastoma cells from which the tumorspheres were developed (200×).

FIG. 2 illustrates U87MG and T98G BTSC glioblastoma and tumorsphere cells derived from human tissue cultures (200×).

FIG. 3 illustrates the expansion of CD133+BTSC's from human glioblastoma cells as tumorspheres in culture.

FIG. 4A provides the structure for the nuclear receptor ligand Butyryl CoA.

FIG. 4B illustrates pictorially represents T98G tumorspheres grown in the presence and absence of the nuclear receptor ligand Butyryl CoA.

FIG. 4C provides a plot of the % survival for T98-Glioma cells cultured in the presence of a range of levels of the ligand Butyryl CoA.

FIG. 4D provides a plot of the % survival for U87-Glioma cells cultured in the presence of a range of levels of the ligand Butyryl CoA.

FIG. 4E provides a plot of the % survival for T98-BTSC cells cultured in the presence of a range of levels of the ligand Butyryl CoA.

FIG. 4F provides a plot of the % survival for U87-BTSC cells cultured in the presence of a range of levels of the ligand Butyryl CoA.

FIG. 4G provides a plot of the % survival for T98-CD133+ cells cultured in the presence of a range of levels of the ligand Butyryl CoA.

FIG. 4H provides a plot of the % survival for U87-CD133+ cells cultured in the presence of a range of levels of the ligand Butyryl CoA.

FIG. 5A provides the structure for the nuclear receptor ligand CITCO.

FIG. 5B illustrates pictorially represents T98G tumorspheres grown in the presence and absence of the nuclear receptor ligand CITCO.

FIG. 5C provides a plot of the % survival for T98-Glioma cells cultured in the presence of a range of levels of the ligand CITCO.

FIG. 5D provides a plot of the % survival for U87-Glioma cells cultured in the presence of a range of levels of the ligand CITCO.

FIG. 5E provides a plot of the % survival for T98-BTSC cells cultured in the presence of a range of levels of the ligand CITCO.

FIG. 5F provides a plot of the % survival for U87-BTSC cells cultured in the presence of a range of levels of the ligand CITCO.

FIG. 5G provides a plot of the % survival for T98-CD133+ cells cultured in the presence of a range of levels of the ligand CITCO.

FIG. 5H provides a plot of the % survival for U87-CD133+ cells cultured in the presence of a range of levels of the ligand CITCO.

FIG. 6A provides the structure for the nuclear receptor ligand GW3965.

FIG. 6B illustrates pictorially represents T98G tumorspheres grown in the presence and absence of the nuclear receptor ligand GW3965.

FIG. 6C provides a plot of the % survival for T98-Glioma cells cultured in the presence of a range of levels of the ligand GW3965.

FIG. 6D provides a plot of the % survival for U87-Glioma cells cultured in the presence of a range of levels of the ligand GW3965.

FIG. 6E provides a plot of the % survival for T98-BTSC cells cultured in the presence of a range of levels of the ligand GW3965.

FIG. 6F provides a plot of the % survival for U87-BTSC cells cultured in the presence of a range of levels of the ligand GW3965.

FIG. 6G provides a plot of the % survival for T98-CD133+ cells cultured in the presence of a range of levels of the ligand GW3965.

FIG. 6H provides a plot of the % survival for U87-CD133+ cells cultured in the presence of a range of levels of the ligand GW3965.

FIG. 7A provides the structure for the nuclear receptor ligand melatonin.

FIG. 7B illustrates pictorially represents T98G tumorspheres grown in the presence and absence of the nuclear receptor ligand melatonin.

FIG. 7C provides a plot of the % survival for T98-Glioma cells cultured in the presence of a range of levels of the ligand melatonin.

FIG. 7D provides a plot of the % survival for U87-Glioma cells cultured in the presence of a range of levels of the ligand melatonin.

FIG. 7E provides a plot of the % survival for T98-BTSC cells cultured in the presence of a range of levels of the ligand melatonin.

FIG. 7F provides a plot of the % survival for U87-BTSC cells cultured in the presence of a range of levels of the ligand melatonin.

FIG. 7G provides a plot of the % survival for T98-CD133+ cells cultured in the presence of a range of levels of the ligand melatonin.

FIG. 7H provides a plot of the % survival for U87-CD133+ cells cultured in the presence of a range of levels of the ligand melatonin.

FIG. 8A provides the structure for the nuclear receptor ligand rifampicin.

FIG. 8B illustrates pictorially represents T98G tumorspheres grown in the presence and absence of the nuclear receptor ligand rifampicin.

FIG. 8C provides a plot of the % survival for T98-Glioma cells cultured in the presence of a range of levels of the ligand rifampicin.

FIG. 8D provides a plot of the % survival for U87-Glioma cells cultured in the presence of a range of levels of the ligand rifampicin.

FIG. 8E provides a plot of the % survival for T98-BTSC cells cultured in the presence of a range of levels of the ligand rifampicin.

FIG. 8F provides a plot of the % survival for U87-BTSC cells cultured in the presence of a range of levels of the ligand rifampicin.

FIG. 8G provides a plot of the % survival for T98-CD133+ cells cultured in the presence of a range of levels of the ligand rifampicin.

FIG. 8H provides a plot of the % survival for U87-CD133+ cells cultured in the presence of a range of levels of the ligand rifampicin.

FIG. 9A provides the structure for the nuclear receptor ligand 15d-PGJ2.

FIG. 9B illustrates pictorially represents T98G tumorspheres grown in the presence and absence of the nuclear receptor ligand 15d-PGJ2.

FIG. 9C provides a plot of the % survival for T98-Glioma cells cultured in the presence of a range of levels of the ligand 15d-PGJ2.

FIG. 9D provides a plot of the % survival for U87-Glioma cells cultured in the presence of a range of levels of the ligand 15d-PGJ2.

FIG. 9E provides a plot of the % survival for T98-BTSC cells cultured in the presence of a range of levels of the ligand 15d-PGJ2.

FIG. 9F provides a plot of the % survival for U87-BTSC cells cultured in the presence of a range of levels of the ligand 15d-PGJ2.

FIG. 9G provides a plot of the % survival for T98-CD133+ cells cultured in the presence of a range of levels of the ligand 15d-PGJ2.

FIG. 9H provides a plot of the % survival for U87-CD133+ cells cultured in the presence of a range of levels of the ligand 15d-PGJ2.

FIG. 10A provides a plot of the in vivo effect for several nuclear receptor agonists on the presence and size of tumors (TG98-G) grown in a live mouse.

FIG. 11B provides a plot of the in vivo effect for several nuclear receptor agonists on the presence and size of tumors (U87MG) grown in a live mouse.

### DESCRIPTION

For the purposes of promoting an understanding of the principles of this disclosure, references will now be made to several embodiments and specific language will be used to describe the same. It will nevertheless be understood that no limitation of the scope of the disclosure is thereby intended, such alterations and further modifications and applications of the principles of the disclosure as described herein being contemplated as would normally occur to one skilled in the art to which the disclosure relates.

This disclosure relates to in vitro methods for identifying nuclear receptor/ligand combinations which are effective for targeting cancerous stem cells and in vivo methods for utilizing the combination to inhibit proliferation of a related cancerous tumor in a mammalian organism. The work completed at this time has focused on BTSC's and the treatment of brain tumors, but is applicable to other forms of cancer having a cancerous form of stem cells which express altered amounts of a nuclear receptor.

### 1. ISOLATION OF BTSC'S AND THEIR EXPANSION AS TUMORSPHERES

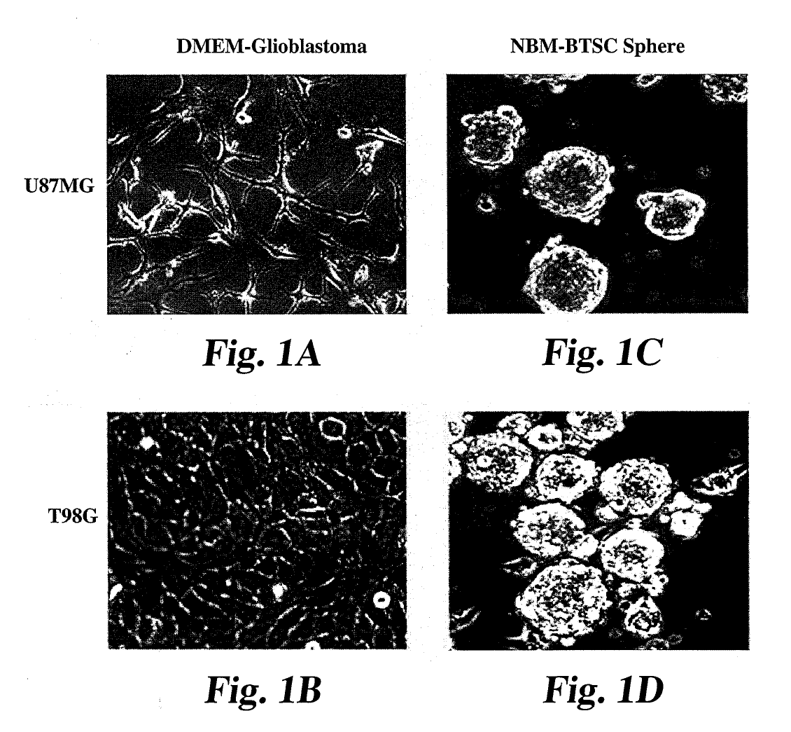

Initially culture conditions were established for expanding BTSC's as tumorspheres from brain tumor cells in culture. Tumorsphere formation resulted in 3 to 5 days when U87MG and T98G human glioblastoma cells were cultured in neurobasal medium (NBM) with B27 supplement in the presence of EGF+bFGF. The resulting tumorspheres increased substantially in size within 7 to 10 days. FIG. 1 illustrates typical glioblastoma cells cultured in DMEM supplemented with 10% FBS and the corresponding tumorspheres cultured in neurobasal medium (NBM) with B27 supplement and 10 ng/ml EGF+bFGF. The tumorspheres illustrated in FIG. 1 were photographed after 10 days (200×).

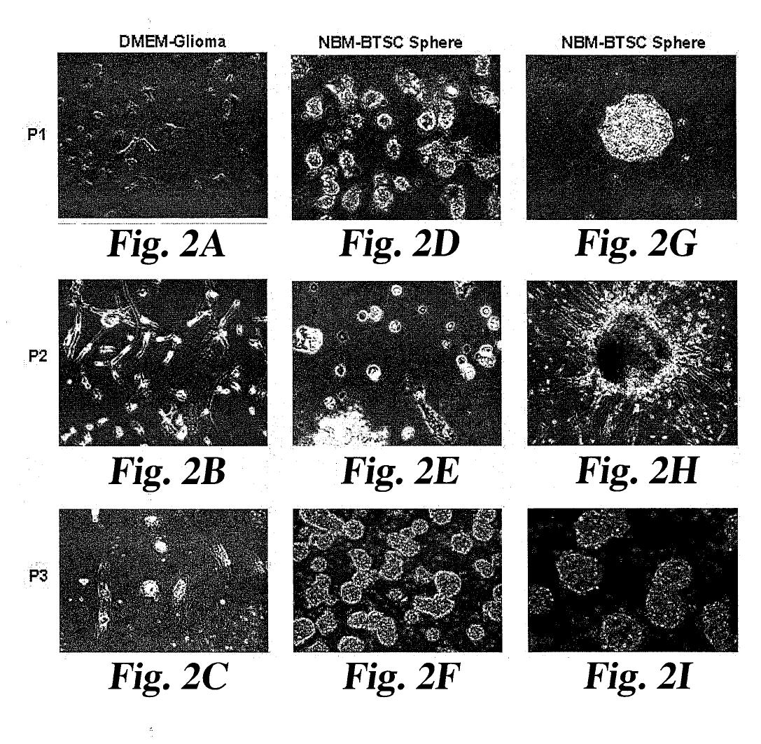

Tumorspheres were also obtained from primary brain tumors. Samples of primary human glioma cells from three patients (P1, P2, and P3) were dissociated and cultured in either DMEM+10% FBS or NBM+B27+EGF+FGF. Within 3-5 days tumorspheres formed and increased substantially in size within 7-10 days. FIG. 2 illustrates the three sets (P1, P2, and P3) of cells and the tumorspheres obtained from each culture (200× and 400×) and demonstrate that BTSC exist in primary human glioma and that the BTSC's can be isolated and expanded in culture.

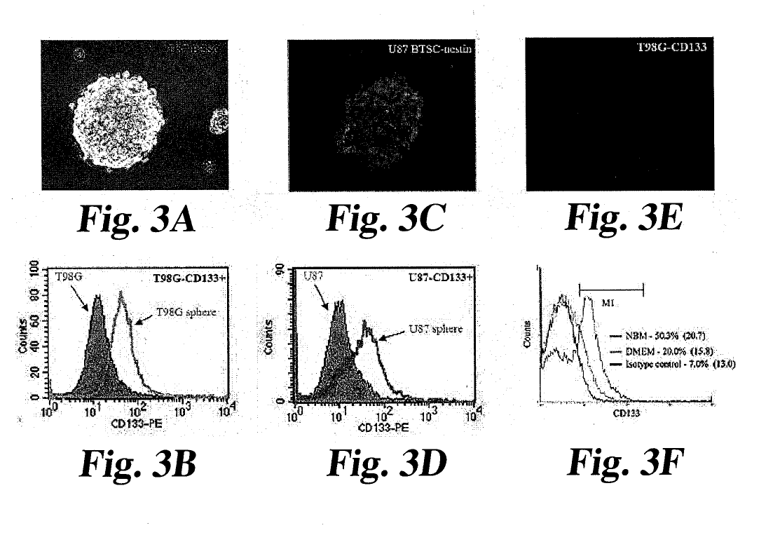

BTSC's were expanded as gliospheres by culturing U87MG or T98G cells in NBM with B27 supplement and EGF+bFGF. After 5 days the gliospheres were stained with anti-nestin antibody conjugated with fluorescen isothiocyanate [a neural stem cell marker] and with anti-CD 133 antibody conjugated with phycoerythrin (PE) [a brain tumor stem cell marker] and photographed utilizing a phase contrast microscope (FIG. 3 left). The gliospheres were dissociated and stained with CD133-PE antibody and analyzed by flow cytometry. The % CD133+ and mean fluorescence intensities are provided in FIG. 3. As noted in FIG. 3 the U87MG cells initially displayed very low levels of CD133+ cells. However, culturing the cells as tumorspheres caused the levels to quickly increase to 50%. The culture conditions developed proved satisfactory to expand the CD133+ BTSC's as tumorspheres which were utilized in the current studies.

### 2. IDENTIFICATION OF NUCLEAR RECEPTORS HAVING ALTERED EXPRESSION IN CANCEROUS STEM CELLS

BTSC's from T98G human glioma were isolated and expanded as tumorspheres in culture. The expression levels of 45 nuclear receptors in BTSC's were analyzed using real time RT-PCR and compared to the receptors expressed in T98G glioma cells. Six nuclear receptors (HNF4α, TLX, PNR, RORα, RORγ, PXR) increased more than 200 fold, 17 nuclear receptors increased more than 10 fold, and 2 nuclear receptors (DAX1, CARα) decreased more than 10 fold in BTSC compared to the T98G cells. Selected results are provided below in Table 1 below.

TABLE 1
Expression profile of nuclear receptors in brain tumor stem cells

| Nuclear receptor | Known Function | Fold Increase | Gene access No |
|---|---|---|---|
| HNF4a (NR2A2) | Expressed in liver, activates lipid, | 4412 | NM_178849 |
| | glucose, vitamin transporter genes | | |
| PNR (NR2E3) | Plays a role in retinal photo | 394 | NM_014249 |
| | receptor cell differentiation and | | |
| | degeneration | | |
| TLX (NR2E1) | Essential for neural stem cell | 382 | NM_003269 |
| | proliferation and self renewal. | | |
| RORα NR1F1) | Play a role in cell survival in | 367 | NM_134260 |
| | central nervous system. | | |
| RORγ NR1F3) | Play a role in thymocyte | 365 | NM_001523 |
| | development | | |
| PXR(NR1I2) | Regulate xenobiotic catabolizing | 245 | NM_033013 |
| | enzyme, reduce bile acid toxicity | | |
| LXRβ NR1H2) | Regulates cholesterol homeostasis | 30 | NM_007121 |
| | in liver and macrophages. | | |
| LXR (NR1H3) | Regulates cholesterol and bile acid | 13 | NM_005693 |
| | metabolism | | |
| PPARγ (NR1C3) | Insulin resistance, hyperlipidemia | 7 | NM_005037 |
| DAXl(NR0Bl) | Expressed in reproductive tissues | 0.0056 | NM_000475 |
| | Inhibits SF-1, ER, AR | | |
| CARα(NR1I3) | Regulates xenobiotic and | 0.017 | NM_005122 |
| | endogenous lipid compounds. | | |

### 3. INHIBITING THE PROLIFERATION OF BTSC'S WITH NUCLEAR RECEPTOR AGONISTS

The use of nuclear receptor agonists in the treatment of brain tumors was examined by testing the effect of the specific ligands on the proliferation of brain tumor and BTSC'S. Prior to studying the anti-tumor effect of melatonin, it was necessary to determine the viability of glioma and BTSC's in the cultures used in this study. T98G and U87MG human glioma cells and the brain tumor stem cells isolated from T98G and U87MG glioma cells were cultured in 96 tissue culture plates (5000 cells/well) at 37° C. incubator temperature without the various agonists for the nuclear receptors and in the presence of different concentrations of nuclear receptor ligands (Butyryl CoA, CITCO, GW3965, Melatonin, Rifampicin, 15d-PGJ2) for 48 hours. Their proliferation was measured by WST-1 assay. The ligands for all six nuclear receptors induced anti-proliferative activity in brain tumor stem cells as well as human glioma cells. It was also noted that the selected nuclear receptor agonists induced anti-tumor activity in brain tumor stem cells and that the different agonists for the highly altered nuclear hormone receptors provided different levels of inhibition for brain tumor cells and BTSC's in cultures. As a result, the nuclear hormone receptors having altered expression and activation profile in BTSC can be selected to specifically target BTSC's. Additional information regarding the individual studies is provided below:

### (i) HNF4α and its Ligand Butyryl CoA:

Hepatocyte nuclear factor α (HNF4α) is a nuclear receptor transcription factor expressed in liver, kidney, intestine and pancreas. HNF4α plays critical roles in liver function. Loss of HNF4α results in liver tumorogenesis and over expression diminished the ability of a hepatocellular carcinoma to proliferate.

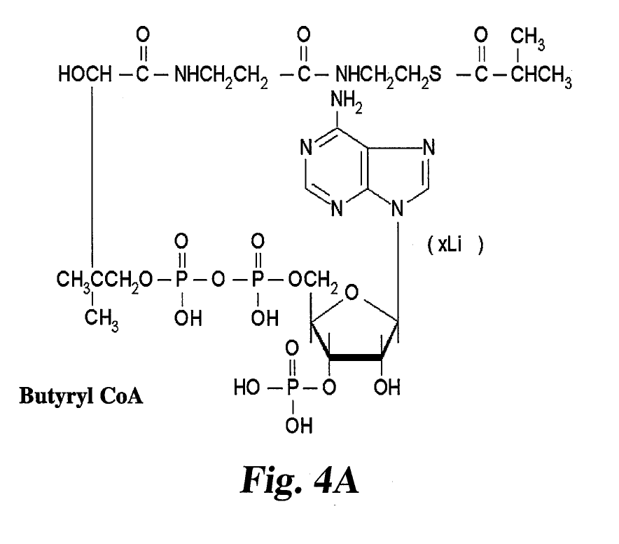

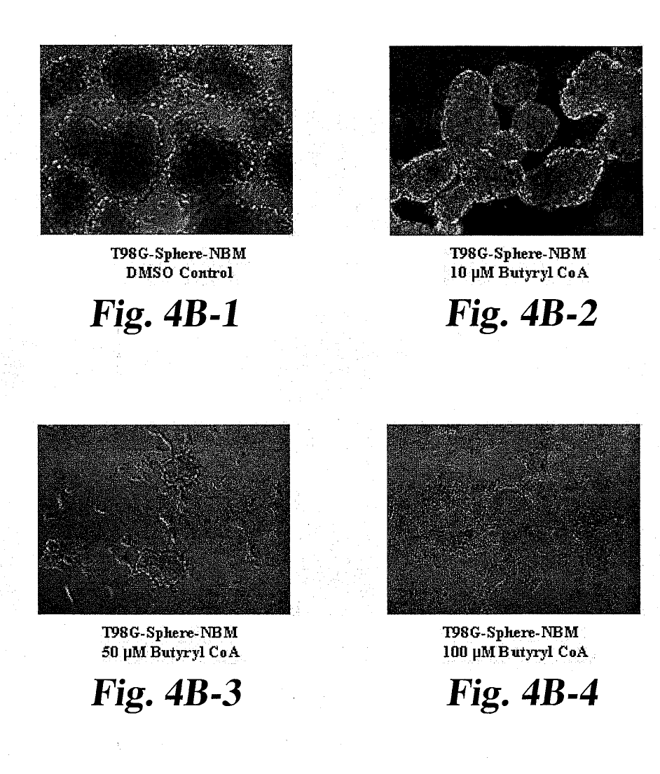

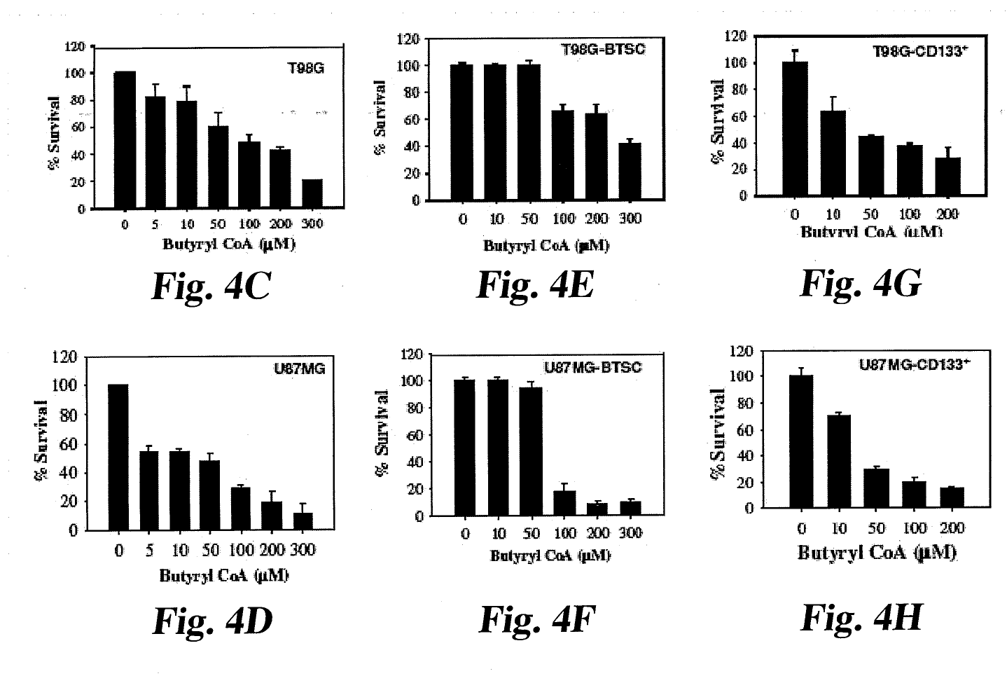

It was further discovered that the expression of HNF4α is significantly increased in BTSC's. Treatment of both U87MG and T98G human glioma cells and the respective BTSC's with Butyryl CoA (FIG. 4A), a ligand for HNFα, resulted in a dose-dependent decrease in the proliferation of the glioma cells and the BTSC's. BTSC's were cultured in NBM medium to expand the stem cells as tumorspheres. Cultures of T98G human glioblastoma cells in neurobasal medium (NBM) with B27 supplement in the presence of EGF+bFGF resulting in tumorsphere formation in 3 to 5 days (see FIG. 4B). Treatment with the HNF4α agonist, Butyryl CoA resulted in a significant and dose-dependent decrease in the number and size of T98G tumorspheres in NBM, indicating that an agonist for a nuclear receptor can inhibit tumorsphere formation and expansion of BTSC from glioma cells in culture. The results from this experiment further indicate that Butyryl CoA induces a dose-dependent inhibition of U87MG and T98G glioma cells and human BTSC's, and purified CD133+ human BTSC's in culture (see FIGS. 4C through 4H).

### (ii) CARα and its Ligand CITCO:

Constitutive androstane receptor (CAR) is an orphan nuclear receptor family of transcription factors that act as a xenosensor and activates target genes (enzymes) responsible for catabolic clearance of toxic compounds. CAR is retained in the cytoplasm complexed with phosphatase 2A (PP2A), HSP90 and Cytosolic CAR retention protein (CCRP). CITCO is a human CAR ligand that induces translocation of CAR to the nucleus from the cytoplasm. CAR binds to the response elements as monomer or as CAR/RXR heterodimer and regulates the synthesis of cholesterol and increases lipolysis in the liver.

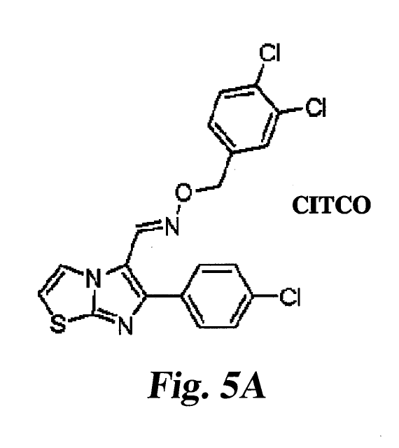

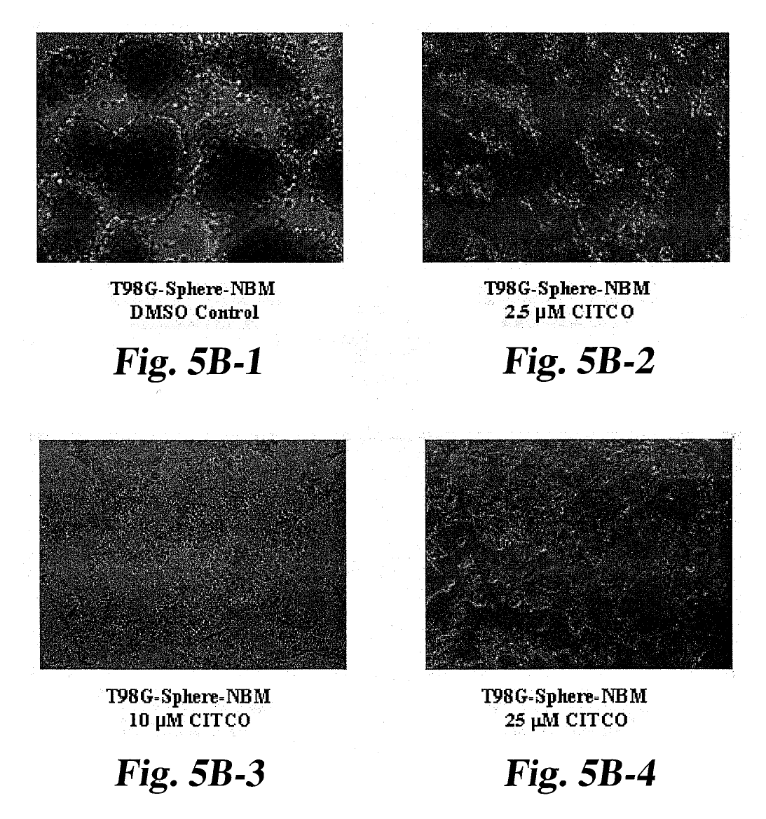

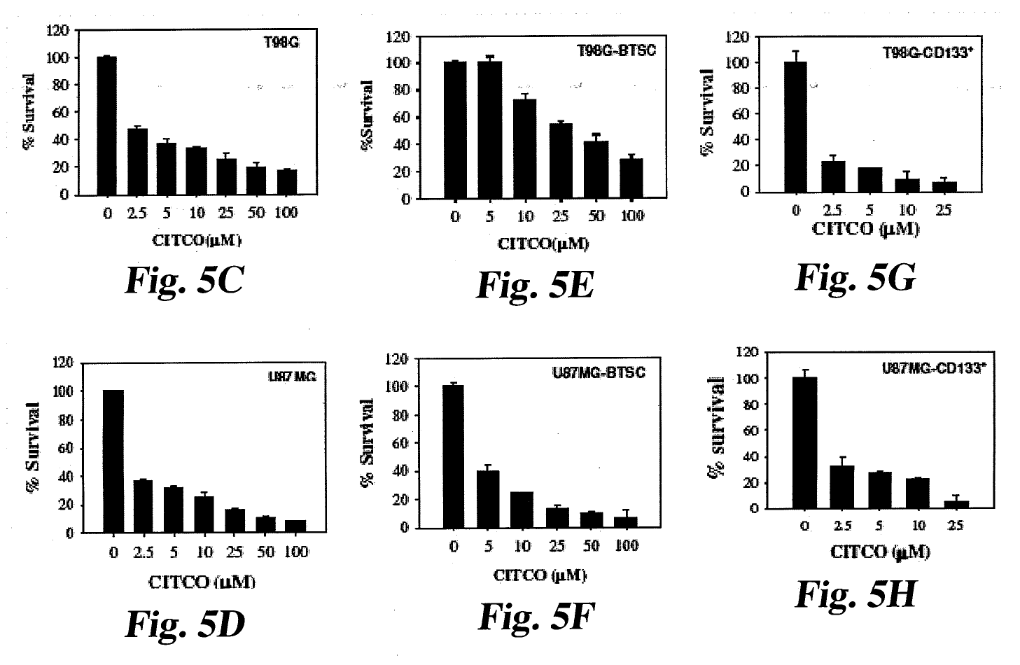

It has been discovered that the expression of CAR is significantly reduced in BTSC's. Treatment with CITCO (FIG. 5A), a specific ligand for human CAR inhibits the proliferation of U87MG and T98G human glioma cells, their respective BTSC's, and the corresponding human CD133 cells (See FIGS. 5B through 5H. These results indicate that CAR ligands such as CITCO can function as potent therapeutic agents to target BTSC's alone or in conjunction with other treatment methods to inhibit their expansion in culture.

As illustrated in FIG. 5B, cultures of T98G human glioblastoma cells in neurobasal medium (NBM) with B27 supplement in the presence of EGF+bFGF resulted in tumorsphere formation in 3 to 5 days. Treatment with the CARα agonist, CITCO resulted in a significant and dose-dependent decrease in the number and size of the T98G tumorspheres cultured in DMEM medium. As illustrated in FIGS. 5C through 5H, CITCO is able to induce a dose-dependent inhibition of cell survival for U87MG glioma cells, T98G glioma cells, human BTSC's and purified CD133+ human BTSC's in culture.

(iii) LXR and its Ligand GW3965

Live X receptors (LXRβ and LXRα) are nuclear receptor transcription factors that regulate cholesterol, glucose and fatty acid metabolism in liver and macrophages. Inactivation of LXR β in mice leads to motor neuron degeneration. Certain LXR agonists have been shown to suppress proliferation of prostate and breast cancer cells in vitro and suppress the growth of prostate tumor xenografts in mice. LXR agonists can also inhibit the inflammatory response in microglia and astrocytes. GW3965 (Benzeneacetic acid, 3-[3-[2-chloro-3-(trifluoromethyl)phenyl]methyl](2,2-diphenylethyl)amino]propoxy]-(9CI) is a ligand for LXR.

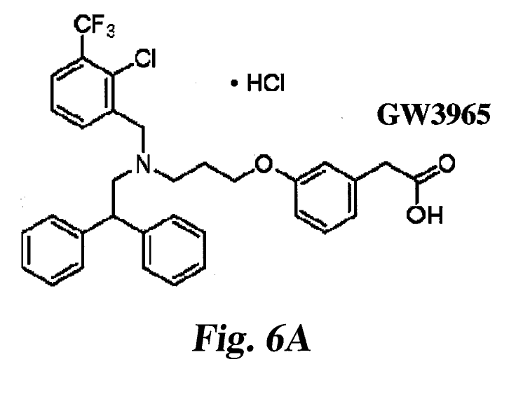

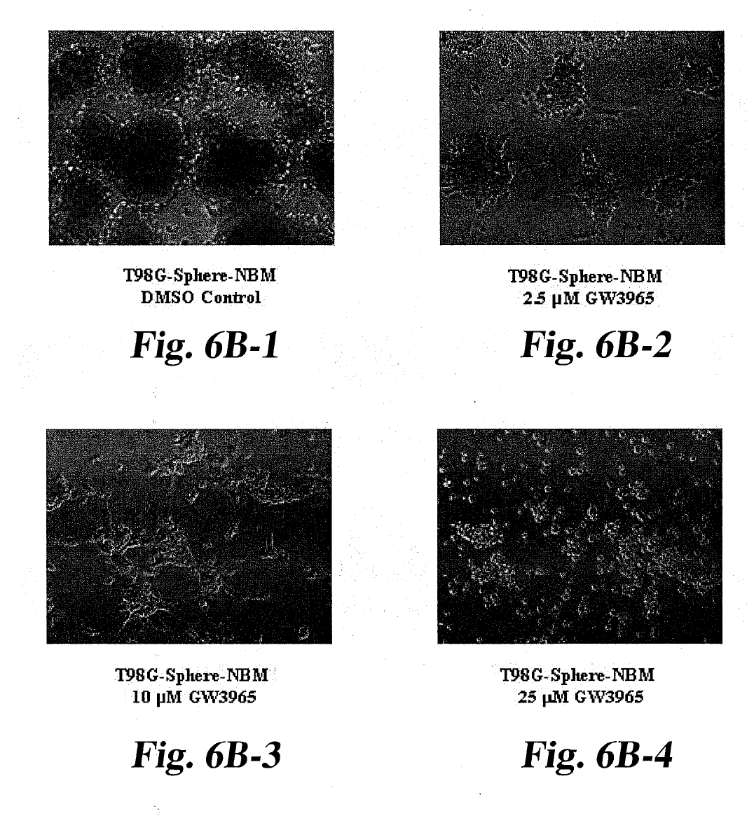

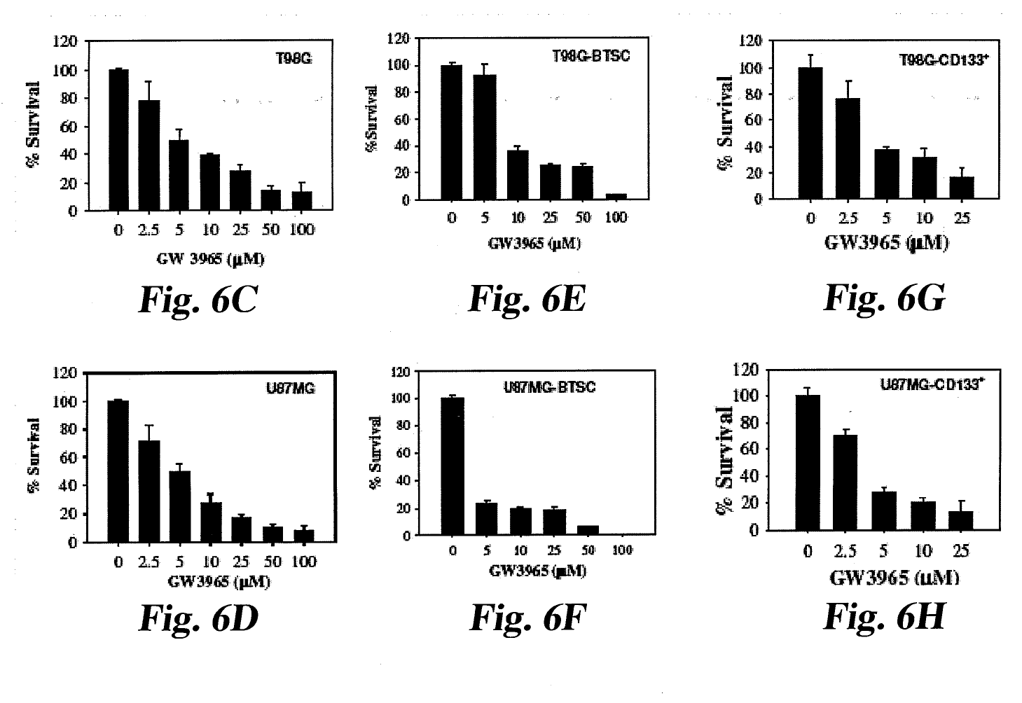

As in the other examples, BTSC's were first cultured under conditions to expand BTSC as tumorspheres from brain tumor cells. As shown in FIG. 6B, culture of T98G human glioblastoma cells in neurobasal medium (NBM) with B27 supplement in the presence of EGF+bFGF resulted in tumorsphere formation in 3 to 5 days. It was observed that treatment with LXR agonist, GW3965, resulted in a significant and dose-dependent decrease in the number and size of T98G tumorspheres in NBM. The morphological features of cells treated with these nuclear receptor agonists appear similar to cells cultured in DMEM medium. These results demonstrate that the nuclear receptor agonist GW3965 inhibits tumorsphere formation and expansion of BTSC from glioma cells in culture.

It has now been demonstrated that the expression of LXR is substantially increased in BTSC's and that GW3965 (FIG. 6A), a specific ligand for human LXR, inhibits the proliferation of U87MG and T98G human glioma cells, their respective BTSC's, and their CD133+ cells (see FIGS. 6C through 6H). These results indicate that LXR ligands such as GW3965 are potent therapeutic agents to target BTSC's and inhibit their expansion in culture.

### (iv) ROR and its Receptor Melatonin:

The orphan nuclear receptor (ROR) related to the retinoic acid nuclear receptor (RAR) is an orphan nuclear receptor that regulates thymocyte development and cell survival. RORα/γ is expressed in embryonic stem cells, infant brain, renal cancer, and colon cancer. ROR mRNA was expressed in parathyroid, testis, uterus, and also in diffuse type of gastric cancer. Melatonin is a specific ligand for ROR.

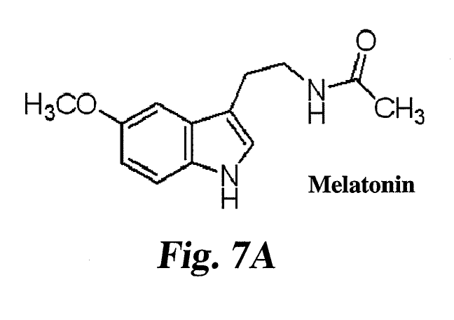

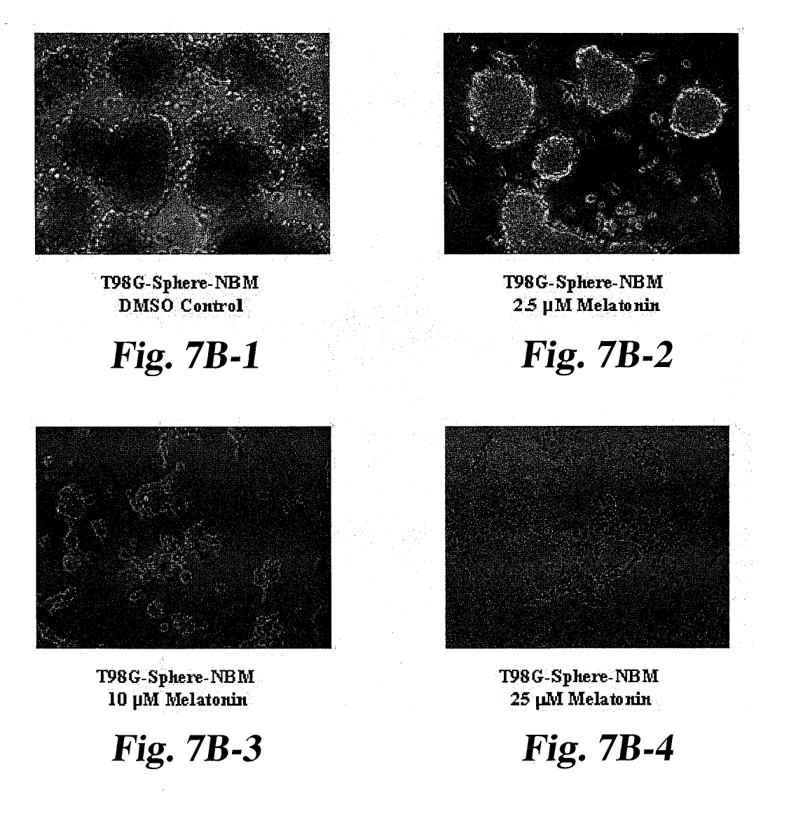

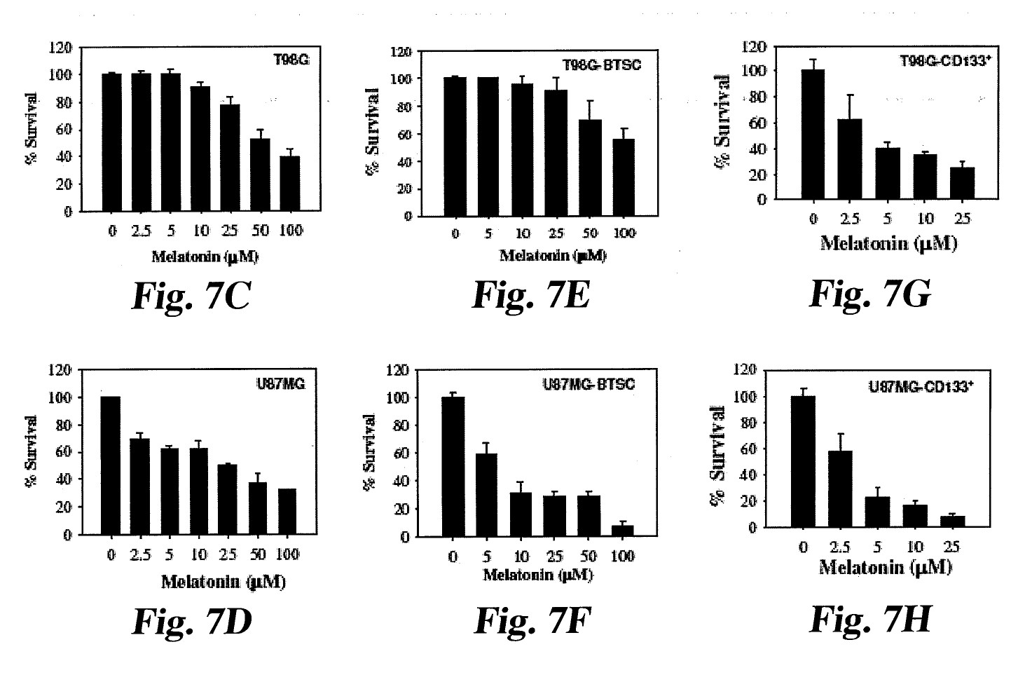

As noted above, it has now been demonstrated that the expression of ROR α/γ is significantly increased in BTSC's. As in the previous study, BTSC's were first cultured under conditions to expand BTSC as tumorspheres from brain tumor cells. As shown in FIG. 7B, culture of T98G human glioblastoma cells in neurobasal medium (NBM) with B27 supplement in the presence of EGF+bFGF resulted in tumorsphere formation in 3 to 5 days. As in the related studies, treatment with LXR agonist, melatonin, resulted in a significant and dose-dependent decrease in the number and size of T98G tumorspheres in NBM. The morphological features of cells treated with these nuclear receptor agonists appear similar to cells cultured in DMEM medium. These results demonstrate the ability of the nuclear receptor agonist, melatonin, to inhibit tumorsphere formation and expansion of BTSC from glioma cells in culture.

Treatment of a cell culture with melatonin (FIG. 7A), a specific ligand for human ROR, inhibits the proliferation of U87MG and T98G human glioma cells, their respective BTSC's, and related CD133+ cells (see FIGS. 7C through 7H). These results indicate that ROR ligands such as melatonin are potent therapeutic agents to target BTSC's in the treatment of brain tumors.

### (v) PXR and its Ligand Rifampicin:

Pregnant X receptor (PXR) is a member of the orphan nuclear hormone receptor family, which acts as a xenosensor. This nuclear receptor is involved in transcriptional induction of hepatic xenobiotic catabolizing enzymes. Human pregnant X receptor (hPXR) is known for its activation by many important clinical drugs, and interacts with many cellular signaling pathways during carcinogenesis. Rifampicin is a ligand for the PXR nuclear receptor.

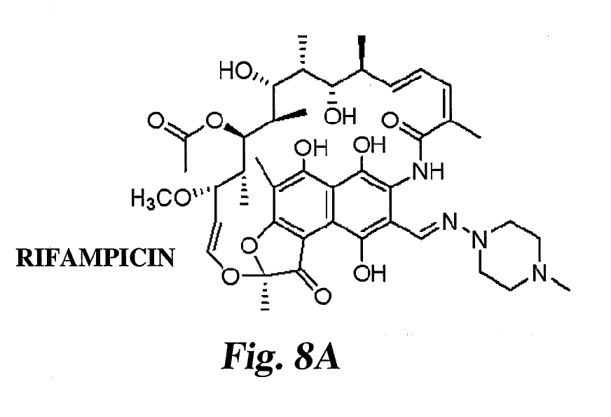

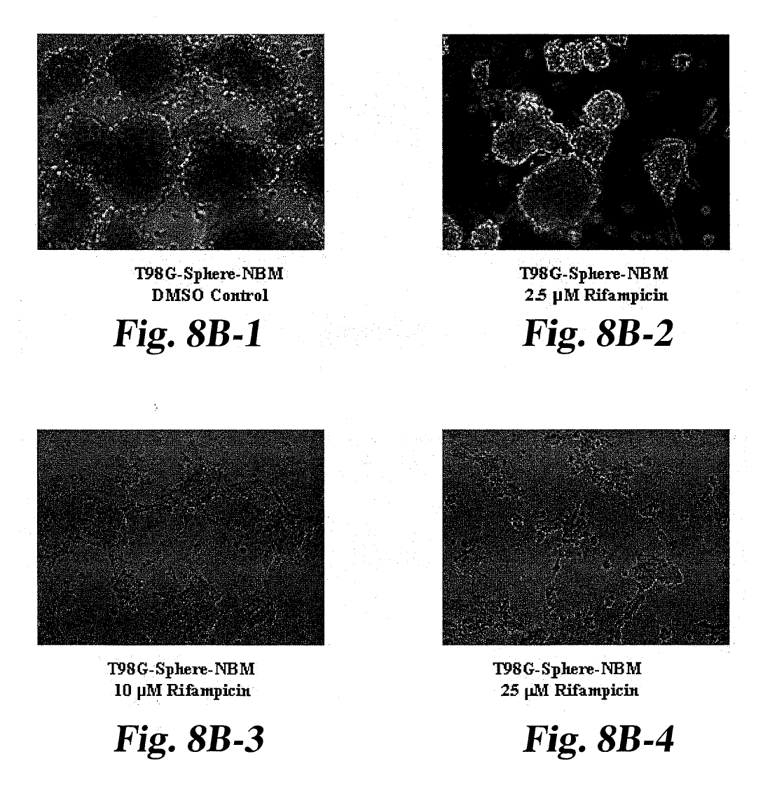

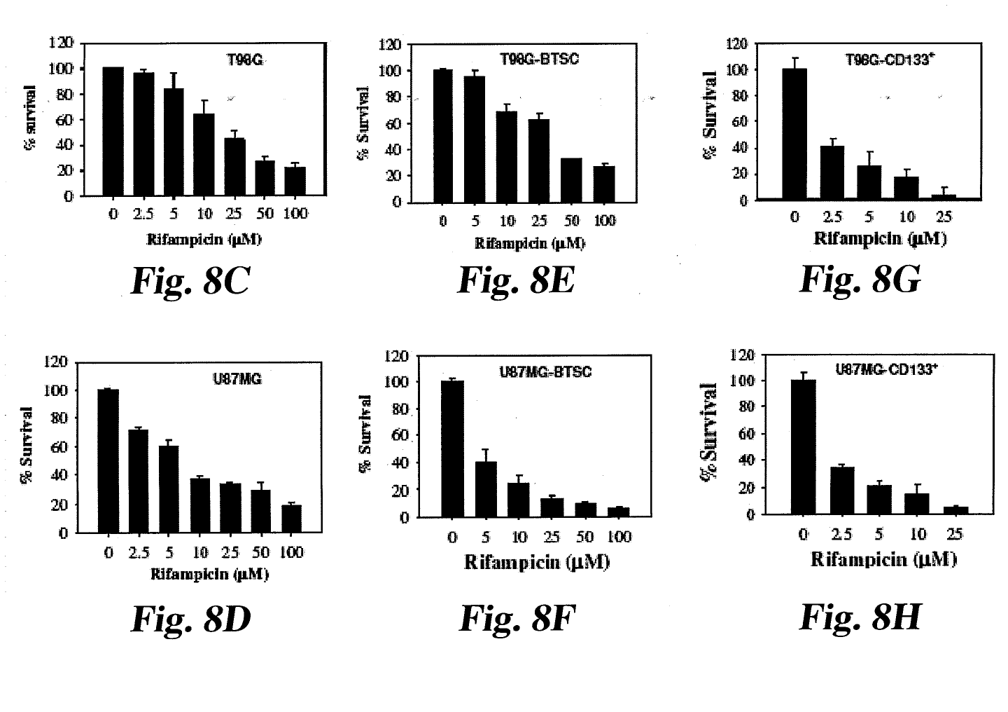

It has been demonstrated that the expression of PXR is significantly increased in BTSC's. Treatment with Rifampicin (FIG. 8A), a specific ligand for human PXR inhibit the proliferation of U87MG and T98G human glioma cells, their respective BTSC's, and related CD133+ cells (see FIGS. 8C through 8H). These results indicate that PXR ligands, such as Rifampicin, are capable of inhibiting tumorsphere formation and the expansion of BTSC's from glioma cells in culture.

(vi) PPARγ and its Ligand 15dPGJ2:

Peroxisome proliferator-activated receptor (PPAR) is a member of the family of nuclear receptor transcription factors which includes PPARα, PPARγ and PPARδ as three known subtypes. PPARγ is expressed in many different tissues and regulates lipid metabolism, glucose homeostasis, tumor progression and inflammation. The 15-deoxy Δ^{12,14}-prostaglandin J2 (15d-PGJ2) is a natural ligand and thiazolidinediones (TZD) such as rosiglitazone is a synthetic agonists for PPARδ. Upon activation with specific ligands, PPARγ heterodimerizes with RXR and induces gene expression associated with cell growth and differentiation. PPARγ agonists regulate adipogenesis and prevent obesity; modulate glucose metabolism and insulin sensitivity thereby reduce plasma glucose and insulin levels in type 2 diabetes. PPARγ agonists also attenuate the clinical symptoms of colitis, arthritis, atherosclerosis, myocarditis, sepsis and multiple sclerosis in animal models.

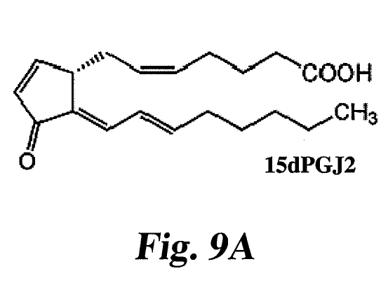

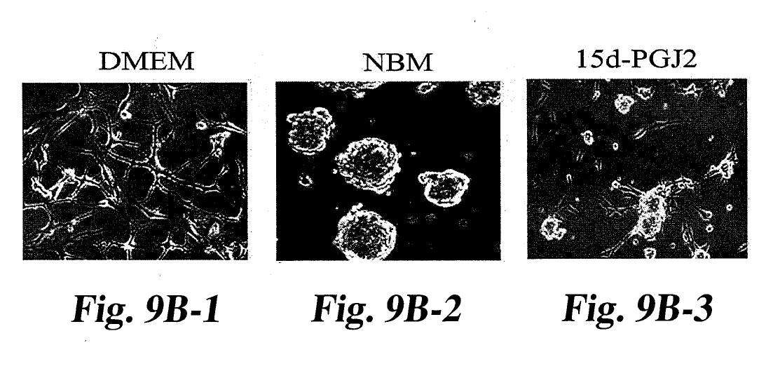

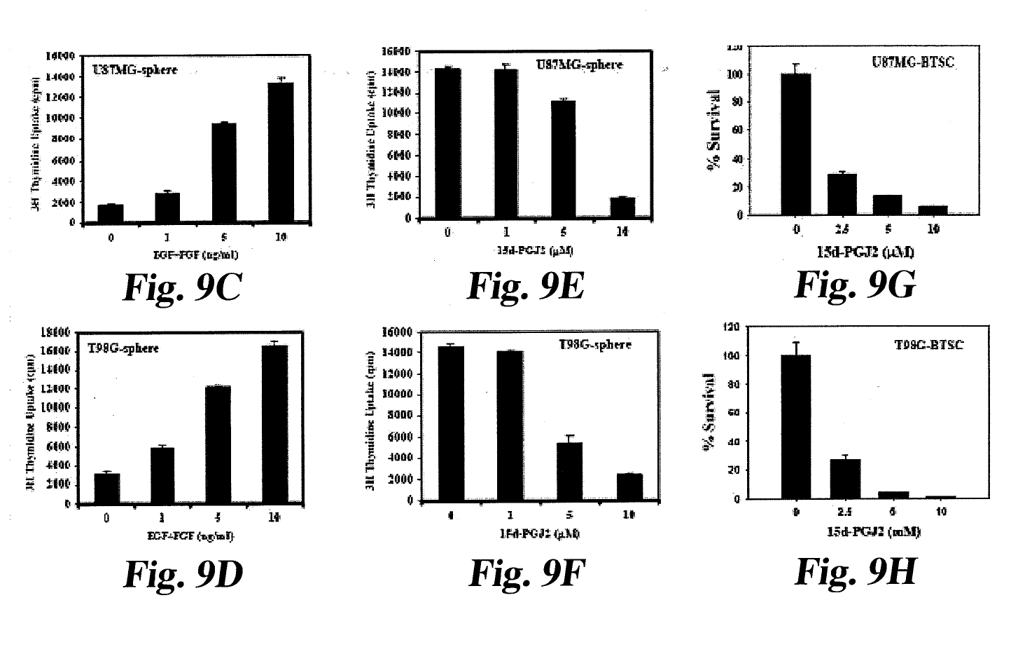

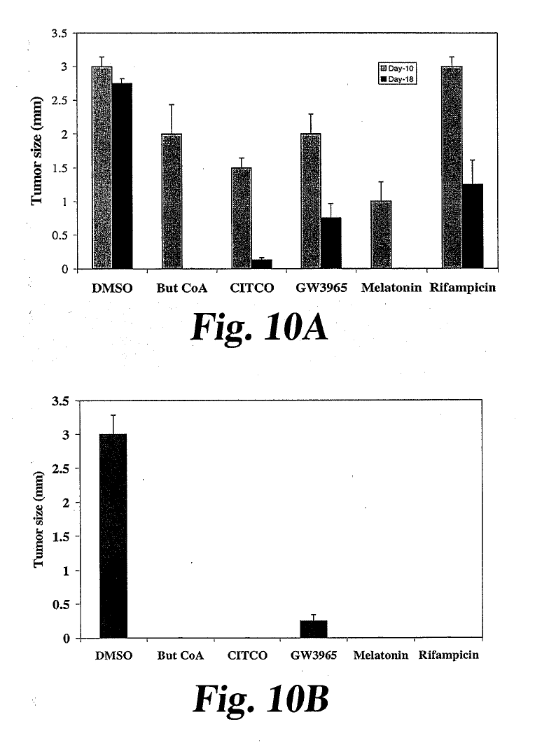

It has been demonstrated that the expression of PPARγ is significantly increased in BTSC's. Treatment with 15d-PGJ2 (FIG. 9A), a specific ligand for human PPARγ inhibited the proliferation of U87MG and T98G human glioma cells. FIG. 9B, first panel, illustrates cells of U87MG cultured in DMEM. Cells cultured in NBM with B27 supplement in the presence of EGF+bFGF developed into tumorspheres within 3-5 days (see FIG. 9A, middle panel). When cultured in the presence of 15d-PGJ2 tumor size and number significantly decreased. The results from cultures maintained in the presence of varying amounts of 15d-PGJ2 is provided in FIGS. 9C through 9H. These results indicate that PPARγ ligands, such as 15d-PGJ2, are capable of inhibiting tumorsphere formation and the expansion of BTSC's from glioma cells in culture.

The results for the selected nuclear receptor/ligand combinations discussed above are provided in Tables 2 and 3, below.

TABLE 2
Nuclear receptor agonists induce anti-tumor activity in T98G
brain tumor cells, T98G BTSC's and T98G-CD133+ cells.

| | Ligand/ | | | |
|---|---|---|---|---|
| Receptor | Agonist | T98-glioma | T98-BTSC | T98G-CD133+ |
| HNF4α | Butyryl CoA | 85.03 ± 5.59 | 85.52 ± 22.44 | 54.71 ± 5.76 |
| CARα | CITCO | 1.37 ± 0.28 | 14.27 ± 2.70 | 3.37 ± 0.30 |
| LXR | GW3965 | 4.85 ± 1.0 | 12.22 ± 3.04 | 3.28 ± 0.64 |
| ROR | Melatonin | 31.04 ± 12.0 | 44.69 ± 6.18 | 2.45 ± 0.43 |
| PXR | Rifampicin | 12.79 ± 1.97 | 22.60 ± 1.94 | 1.99 ± 0.47 |
| PPARγ | 15d-PGJ2 | 12.20 ± 2.17 | 23.50 ± 4.31 | 3.36 ± 0.16 |

*The numbers in the table represent EC50.

TABLE 3
Nuclear receptor agonists induce anti-tumor activity in U87
brain tumor cells, U87 BTSC's and U87-CD133+ cells.

| Receptor | Ligand | U87-glioma | U87-BTSC | U87-CD133+ |
|---|---|---|---|---|
| HNF4α | Butyryl CoA | 20.27 ± 0.57 | 72.50 ± 1.57 | 17.58 ± 0.29 |
| CARα | CITCO | 1.38 ± 0.89 | 2.98 ± 0.18 | 0.53 ± 0.13 |
| LXR | GW3965 | 4.22 ± 0.23 | 1.55 ± 0.43 | 2.96 ± 0.21 |
| ROR | Melatonin | 18.66 ± 0.18 | 7.91 ± 1.34 | 2.6 ± 0.25 |
| PXR | Rifampicin | 25.39 ± 4.0 | 2.97 ± 0.19 | 1.2 ± 0.20 |
| PPARγ | 15d-PGJ2 | 14.20 ± 2.16 | 12.60 ± 1.79 | 1.17 ± 0.09 |

*The numbers in the table represent EC50.

### 4. EFFECTS OF A NUCLEAR RECEPTOR ON BTSC-MEDIATED TUMOR GROWTH IN AN ANIMAL MODEL IN VIVO

Transplantation of T98G and U87MG tumor cells derived BTSC spheres progressed to solid tumors with in 7 days. Tumor size in animals not treated with a nuclear agonist increased by 15 days, whereas, tumors in treated animals significantly decreased the tumor size within 16 days. Under the conditions of this study, the nuclear receptors induced almost complete inhibition of U87MG-sphere derived tumors and substantially inhibited (30-60%) growth of the T98G-BTSC derived tumors. By day 23 of this study a majority of the nuclear receptor agonists induced significant inhibition of tumor progression in nude mice, demonstrating that the nuclear receptor/agonist combination can be an effective for the in vivo treatment of tumors, particularly brain tumors. The results of this study are illustrated in FIGS. 11 and 12.

### 5. MATERIALS AND METHODS

Reagents. The murine recombinant epidermal growth factor (EGF) and basic fibroblast growth factor (bFGF) were purchased from Chemicon International (Temecula, Calif.). 15-Deoxy-Δ^{12,14}-Prostaglandin J₂ (15d-PGJ2) was purchased from Calbiochem (La Jolla, Calif.). All-trans retinoic acid (ATRA) and other chemicals were purchased from Sigma Chemicals Co. (St Louis, Mo.).

Cell culture. The U87MG and T98G brain tumor cell-lines, established from human glioblastoma (Ponten, et al., 1968) were obtained from American Type Culture Collection (ATCC, Manassas, Va.). The cells were cultured in Dulbecco's Modified Eagle Medium (DMEM) (Invitrogen, California, USA) supplemented with 10% FBS, 1 mM Sodium pyruvate, 100 U/ml penicillin G, 100 μg/ml Streptomycin, 2 mM glutamine, 1 mM MEM nonessential amino acids and 50 μM 2-mercaptoethanol in 5% CO₂ incubator at 37° C. The cells were dissociated using 0.25% trypsin, 0.53 mM EDTA solution and sub-cultured once in 3-5 days. Primary brain tumor tissue/cells were also obtained from the tissue repository at Methodist Research Institute and cultured (1×10⁵ cells/ml/well) in DMEM medium supplemented with 10% FBS and sub-cultured once in 10-12 days.

Gliosphere culture. To generate gliospheres, the following standardized method was utilized. Briefly, U87MG and T98G glioblastoma cells were dissociated from DMEM cultures using trypsin-EDTA solution and reseeded in neurobasal medium (NBM) supplemented with B27 in the presence of 10 ng/ml bFGF and EGF. The cells (5×10⁴/ml/well) were cultured in 12 well plates in 5% CO₂-incubator at 37° C. with a medium change of every 2-3 days. The cells were photographed (200× and 400×) under phase contrast microscope (AX70, Olympus Optical, Japan) after 10-12 days. The primary human glioblastoma cells were also dissociated from DMEM using trypsin-EDTA. The cells were cultured (1×10⁵ cells/ml/well) in neurobasal medium (NBM) supplemented with B27 in the presence of 10 ng/ml of bFGF and EGF in 12 well plates in 5% CO₂-incubator at 37° C. The resulting tumorspheres were photographed under light microscope.

Isolation and expansion of CD133+ BTSC: The CD133+ BTSC were isolated from human glioblastoma cells by magnetic bead technique using CD133 antibody (Miltenyi Biotec, Auburn, Calif., USA). The U87MG and T98G glioma cells and tumor spheres expanded as described above were dissociated by treatment with Trypsin-EDTA solution at 37° C. for 10 minutes. The cells (1×10⁷ cells) were washed in cold sterile PBS, re-suspended in 100 μl blocking buffer (PBS, pH 7.2, 0.5% BSA, 2 mM EDTA) with 50 μl of FcR blocking reagents (Miltenyi Biotech, Auburn, Calif., USA) and incubated at 4° C. for 30 minutes. The cells were washed and re-suspended in 200 μl of anti-PE microbeads in the buffer and incubated at 4° C. for 30 minutes. The CD133+ cells were separated by passing through an LS column in the midi Macs separation unit. The column effluent was collected to provide a CD133 negative population. The columns were washed with buffer, removed from the Mac separation unit and placed in a sterile 15 ml tube. The CD133 positive cells bound on the columns were isolated by flashing with the buffer for several cycles. The cells obtained were counted and expanded in culture or used directly for the experiments described herein.

Flow cytometry. Seven to ten days old gliospheres were cultured in 12 well tissue culture plates in NBM supplemented with B-27 and 10 ng/ml EGF+bFGF in 5% CO₂ incubator at 37° C. for 72 hrs. The cells were dissociated, fixed in 1% paraformaldehyde at 4° C. for 20 min and permeabilized with 0.02% Triton X-100 in PBS. After incubation in the blocking buffer (3% BSA in PBS) at 4° C. for 20 min, the cells were stained with anti-CD133 (1:10, Miltenyi Biotec, Auburn, Calif., USA) antibodies at 4° C. for one hr. The cells were washed three times with 0.1% BSA in PBS, incubated with FITC conjugated secondary antibody for 30 min and analyzed by flow cytometry. The gliospheres were also stained as above and photographed under Olympus fluorescence microscope.

Proliferation assay. Proliferation of gliosphere cells was measured using WST-1 assay (Roche Applied Sciences, Indianapolis, Ind.) and ³H thymidine uptake assay. Briefly, T98G and U87MG glioma and gliosphere cells were cultured in 96-well tissue culture plates (1×10⁴/200 μl/well) in neurobasal medium with B27 serum supplement in the presence of 10 ng/ml EGF+bFGF and increasing doses of nuclear receptors in 5% CO₂-incubator at 37° C. After 48 hrs, WST-1 reagent (101/well) was added and the absorbance was measured at 460 nm using 2100 microplate reader (Alpha Diagnostics Inc., San Antonio, Tex.) as a measure of viable cell count. This assay is based on the cleavage of the tetrazolium salt, WST-1, by mitochondrial dehydrogenases in viable cells and a decrease in OD corresponds to decrease in viable cell count. In some experiments, ³H thymidine (0.5 μCi/ml) was added at 48 hours, the cells harvested after 72 hours using a Tomtech harvester 96 (Hamden, Conn., USA) and the amount of ³H thymidine uptake counted on a Perkin Elmer Microbeta liquid scintillation counter to provide a measure of proliferation.

Quantitative reverse transcription polymerase chain reaction. The T98G glioma cells were cultured in DMEM with 10% FBS. The T98G-derived BTSC were expanded as tumorspheres by culturing (2×10⁶ cells/well) in NBM+B27 with 10 ng/ml EGF+bFGF. After 5 days, the glioma and BTSC spheres were harvested and the total RNA was extracted using TRIzol reagent according to standard protocol. The cDNA was reverse transcribed by incubating 5 μg of total RNA in 10 μl reaction of random hexamer primers and master mix from TaqMan reverse transcription kit (Applied Biosystems, Branchburg, N.J.). For quantitative real-time PCR, 2 μl of the cDNA (equivalent to 0.1 μg total RNA) was amplified in TaqMan Universal Master Mix with nuclear receptor primer sets and probes in an optical 384-well reaction plate using the 7900 Fast Sequence Detection Real-time PCR System (Applied Biosystems, Foster City, Calif.). The results were analyzed using the Prism 7900 relative quantification (delta delta Ct) study software (Applied Biosystems, Foster City, Calif.). The level of nuclear receptor gene expression was normalized to GAPDH and expressed as arbitrary fold change compared to control sample.

In vivo model for brain tumor stem cell. To demonstrate the anti-tumor activity of nuclear receptor agonists in vivo, T98G and U87MG BTSC spheres (0.5×10⁶ cell) in 0.5 ml NBM were injected subcutaneously into 5-6 week old male athymic nude mice. The development of solid tumors was observed in 5-7 days. The mice were treated by injection (into the peritoneum) with 100 μg of a nuclear receptor agonist (dissolved in 25 μl of DMSO) on days 7, 9, 12, 14, 16, 19 and 21 (counting from the day of tumor cell injection). Control mice received only 25 μl of DMSO. The animals were observed every day and tumor size was measured on days 16 and 23 by using Calipers with data presented as a histogram. The tumor samples were dissected out on day 30 and thin sections (10 μm) were stained with H&E using standard protocols and photographed under microscope. The tumor cells were dissociated and the viability/proliferation assayed by WST-1 technique. All procedures were conducted in accordance with Methodist Research Institutes Animal Care and Use Committee guidelines.

Statistical analysis. The ANOVA method was used to determine the statistical significance of the results.

### 6. CONCLUSIONS

The current studies demonstrate that cancerous stem cells provide an altered expression of nuclear receptors and provide methods for identifying which nuclear receptor have an altered expression. Further, methods are provided for determining nuclear receptor/ligand combinations which can inhibit the proliferation and survival of cancerous stem cells. Finally, methods are provided for utilizing a combination of the identified nuclear receptors and a corresponding ligand to treat a mammalian tumor or cancerous growth. The methods can be used in vitro or in vivo and are particularly suitable for affecting the growth and survival of human brain tumors. The methods for treating mammalian tumors can be used alone or in conjunction with other forms of cancer treatment such as radiation, surgery, chemotherapy and the like. When fully optimized, treatments directed to regular cancer cells as well as tumor stem cells will provide means to increase both survival times and survival rates for individuals with brain tumors and other forms of cancerous tumors.

While applicant's invention has been described in detail above with reference to specific embodiments, it will be understood that modifications and alterations in embodiments disclosed may be made by those practiced in the art without departing from the spirit and scope of the disclosure. All such modifications and alterations are intended to be covered. In addition, all publications cited herein are indicative of the level of skill in the art and are hereby incorporated by reference in their entirety as if each had been individually incorporated by reference and fully set forth.

1. A method for treatment of a tumor, the method comprising:
obtaining a sample of tumor cells from a subject possessing said tumor;
isolating tumor stem cells from the sample of tumor cells;
analyzing the tumor stems cells for expression levels of nuclear receptors;
identifying a nuclear receptor which has said expression level;
identifying an agonist for said nuclear receptor; and
treating said subject with said agonist.

2. The method of claim 1, wherein said obtaining involves obtaining a sample of tumor cells from a brain tumor.

3. The method of claim 2, wherein said identifying a nuclear receptor involves identifying a nuclear receptor selected from the group consisting of HNF4α, PNR, TLX, RORα, RORγ, PXR, LXRβ, LXR, PPARγ, DAX1, and CARα.

4. The method of claim 2, wherein said identifying a nuclear receptor involves identifying a nuclear receptor selected from the group consisting HNF4α, PNR, TLX, PXR, LXRβ, LXR, DAX1, and CARα.

5. The method of claim 2, wherein said identifying a nuclear receptor involves identifying a nuclear receptor selected from the group consisting of HNF4α, RORα, RORγ, PXR, LXRβ, LXR, PPARγ, and CARα.

6. The method of claim 5, wherein said identifying an agonist involves identifying an agonist selected from the group consisting of Butyryl CoA, CITCO, GW3965, Melatonin, Rifampicin, and 15d-PGJ2.

7. The method of claim 2, wherein said identifying a nuclear receptor involves identifying a nuclear receptor selected from the group consisting HNF4α, PXR, LXRβ, LXR, and CARα.

8. The method of claim 7, wherein said identifying an agonist involves identifying an agonist selected from the group consisting of Butyryl CoA, CITCO, GW3965, and Rifampicin.

9. The method of claim 1, wherein said treating with said agonist involves placing said agonist at the site of the tumor.

10. A method for treatment of a subject with a brain tumor which contains brain tumor stem cells, the method comprising:
identifying a nuclear receptor expressed by the brain tumor stem cells;
identifying an agonist of said nuclear receptor expressed by brain tumor stem cells; and
treating said subject with said agonist.

11. The method of claim 10, wherein said identifying a nuclear receptor involves identifying a nuclear receptor selected from the group consisting of HNF4α, PNR, TLX, RORα, RORγ, PXR, LXRβ, LXR, PPARγ, DAX1, and CARα.

12. The method of claim 10, wherein said identifying a nuclear receptor involves identifying a nuclear receptor selected from the group consisting of HNF4α, RORα, RORγ, PXR, LXRβ, LXR, PPARγ, and CARα.

13. The method of claim 12, wherein identifying said agonist involves identifying an agonist selected from the group consisting of Butyryl CoA, CITCO, GW3965, Melatonin, Rifampicin, and 15d-PGJ2.

14. The method of claim 10, wherein said identifying a nuclear receptor involves identifying a nuclear receptor selected from the group consisting of HNF4α, PXR, LXRβ, LXR, and CARα.

15. The method of claim 14, wherein identifying said agonist involves identifying an agonist selected from the group consisting of Butyryl CoA, CITCO, GW3965, and Rifampicin.

16. The method of claim 10, wherein said treating with said agonist involves placing said agonist at the site of the tumor.

17. An in vitro method of identifying a substance to treat tumors, the method comprising:
obtaining a sample of tumor stem cells from a subject possessing a tumor;
identifying in the sample of tumor stem cells an expression level of a nuclear receptor; and
identifying a ligand of said nuclear receptor identified in said sample of tumor stem cells wherein said ligand is the substance identified to treat the tumor.

18. The method of claim 17, wherein said obtaining involves obtaining a sample of stem cells from a brain tumor.

19. An in vitro method for identifying nuclear receptor/ligand combinations which can be utilized to target cancerous stem cells, the method comprising:
identifying a nuclear receptor expressed by the cancerous stem cell;
selecting a ligand expected to coordinate with the selected nuclear receptor;
culturing the cancerous stem cells in the presence of the selected ligand; and
observing the level of inhibition obtained in the presence of the selected ligand, wherein an inhibition of the cancerous stem cells indicates that the ligand can be utilized to target the cancerous stem cells.

20. The method of claim 19, wherein identifying a nuclear receptor expressed by the cancerous stem cell involves identifying a nuclear receptor expressed by a cancerous brain tumor stem cell.

21. An in vivo method of treating brain tumor stem cells, the method comprising:
identifying a patient with a brain tumor wherein said brain tumor possesses brain tumor stem cells; and
administering to said patient a material from the group consisting of Butyryl CoA, CITCO, GW3965, Melatonin, Rifampicin, and 15d-PGJ2.

22. The method of claim 21, wherein administering to said patient involves administering a material is selected from the group consisting of Butyryl CoA, CITCO, GW3965, and Rifampicin.

23. The method of claim 21, where administering to said patient involves administering said material by ingestion.

24. The method of claim 21, wherein administering to said patient involves administering said material at the site of said tumor.

25. An in vivo method for treating a mammalian organism having a cancerous growth or tumor, the method comprising:
identifying a ligand associated with a nuclear receptor having altered expression in a cancerous stem cell; and
administering a pharmaceutical formulation containing the ligand to the organism.

26. The method of claim 25, wherein administering a pharmaceutical formulation involves administering said pharmaceutical formulation by injection, osmotic pump, IV administration, ingestion, dermal application, or inhalation.

27. The method of claim 25, wherein identifying said ligand involves identifying a ligand selected from the group consisting of Butyryl CoA, CITCO, GW3965, Melatonin, Rifampicin, and 15d-PGJ2.

28. The method of claim 25, wherein identifying said ligand involves identifying a ligand selected from the group consisting of Butyryl CoA, CITCO, GW3965, and Rifampicin.

29. A method for identifying nuclear receptors having an altered expression in a form of cancerous stem cells, the method comprising:
obtaining a culture of regular cancer cells;
obtaining a culture of the related cancerous stem cells from the regular cancer cells;
culturing both sets of cells separately,
determining a level of expression of a selected nuclear receptor in both cultures; and
comparing the level of expression of the selected nuclear receptor by the cancerous stem cell compared to the regular cancer cells wherein a difference in the level of expression indicates the nuclear receptor having an altered expression.

30. The method of claim 29, wherein obtaining a culture of regular cancer cells involves obtaining Glioblastoma cells.
